(19) United States
(12) Patent Application Publication
(10) Pub. No.: US 2011/0224544 A1
(43) Pub. Date: Sep. 15, 2011

(54) ULTRASONIC DIAGNOSTIC APPARATUS WITH VARIABLE ELEVATION TYPE CONTROL PANEL

(75) Inventors: June Young AHN, Seoul (KR); Jae Yong Ko, Seoul (KR)

(73) Assignee: MEDISON CO., LTD.

(21) Appl. No.: 12/967,630

(22) Filed: Dec. 14, 2010

(30) Foreign Application Priority Data
Mar. 15, 2010 (KR) 10-2010-0022861

(51) Int. Cl. A61B 8/00

(52) U.S. Cl. 600/437

(57) ABSTRACT

An ultrasonic diagnostic apparatus with a variable elevation type control panel is disclosed. The ultrasonic diagnostic apparatus with the variable elevation type control panel includes a control panel having a lift operating part, a support supporting the control panel, and a lift connected to the support and operated by manipulation of the lift operating part to lift or lower the support.

### BACKGROUND OF THE INVENTION

1. Field of the Invention

The present invention relates to an ultrasonic diagnostic apparatus and, more particularly, to an ultrasonic diagnostic apparatus that includes a variable elevation type control panel, the height of which can be easily adjusted.

2. Description of the Related Art

Generally, an ultrasonic diagnostic apparatus refers to a non-invasive apparatus that irradiates an ultrasound signal from a surface of a patient body towards a target internal organ beneath the body surface and obtains an image of a monolayer or blood flow in soft tissue from information in the reflected ultrasound signal (ultrasound echo-signal).

The ultrasonic diagnostic apparatus has been widely used for diagnosis of the heart, the abdomen, the urinary organs, and in obstetrics and gynecology due to various merits such as small size, low price, real-time image display, and high stability through elimination of radiation exposure, as compared with other image diagnostic apparatus, such as X-ray diagnostic apparatus, computerized tomography scanners (CT scanners), magnetic resonance imagers (MRIs), nuclear medicine diagnostic apparatuses, and the like.

The ultrasonic diagnostic apparatus includes a probe that transmits an ultrasound signal to an object and receives the ultrasound echo-signal reflected therefrom to obtain an ultrasound image of the object.

The signal received by the probe is output to an image display unit of the apparatus through a controller and an operator examines the object while alternately viewing the monitor and the object.

The display unit is secured to an upper side of a control panel which has an operation button.

Here, it should be noted that the above description is provided for understanding of the background of the invention and is not a description of a well-known conventional technique in the art to which the present invention pertains.

Positional adjustment of the control panel and the display unit is often required depending on conditions for operation. However, since the control panel is secured to a main body of the apparatus, it is inconvenient for an examiner to use the control panel. Therefore, there is a need for an improved ultrasonic diagnostic apparatus that overcomes such a problem.

### SUMMARY OF THE INVENTION

The present invention is conceived to solve the problem of the related art, and an aspect of the invention is to provide an ultrasonic diagnostic apparatus that is provided with a variable elevation type control panel, the height of which can be easily adjusted.

In accordance with an aspect of the invention, an ultrasonic diagnostic apparatus with variable elevation type control panel includes: a control panel having a lift operating part; a support supporting the control panel; and a lift connected to the support and operated by manipulation of the lift operating part to lift or lower the support.

The lift may include a gas spring connected to the lift operating part via a cable and adjusting a lifted or lowered position of the control panel.

The lift may further include a connecting member connected at one side thereof to the support and at the other side thereof to the gas spring.

The ultrasonic diagnostic apparatus may further include a first guide part provided to one side of the connecting member to guide a vertical movement of the connecting member.

The first guide part may include a roller member provided to one side of the connecting member and a rail member disposed along a movement path of the connecting member while surrounding a side surface of the roller member.

The ultrasonic diagnostic apparatus may further include a second guide part adjoining the support and guiding a vertical movement of the support.

### BRIEF DESCRIPTION OF THE DRAWINGS

The above and other aspects, features and advantages of the invention will become apparent from the following description of embodiments given in conjunction with the accompanying drawings, in which:

FIG. 1 is a schematic perspective view of an ultrasonic diagnostic apparatus including a variable elevation type control panel according to one embodiment of the present invention;

FIG. 2 is a schematic perspective view of a lift provided to the ultrasonic diagnostic apparatus according to the embodiment of the present invention;

FIG. 3 is a schematic perspective view of a lift operating part provided to the ultrasonic diagnostic apparatus according to the embodiment of the present invention;

FIG. 4 is a schematic perspective view of the lift before operation according to the embodiment of the present invention;

FIG. 5 is a schematic perspective view of the lift after operation according to the embodiment of the present invention;

FIG. 6 is an exploded perspective view of a rail member and a second guide part shown in FIG. 5; and

FIG. 7 is a plan view of the lift provided to the ultrasonic diagnostic apparatus according to the embodiment of the present invention.

### DETAILED DESCRIPTION OF THE EMBODIMENT

Exemplary embodiments of the invention will now be described in detail with reference to the accompanying drawings. It should be noted that the drawings are not to precise scale and may be exaggerated in thickness of lines or size of components for descriptive convenience and clarity only. Furthermore, terms used herein are defined by taking functions of the invention into account and can be changed according to the custom or intention of users or operators. Therefore, definition of the terms should be made according to overall disclosures set forth herein.

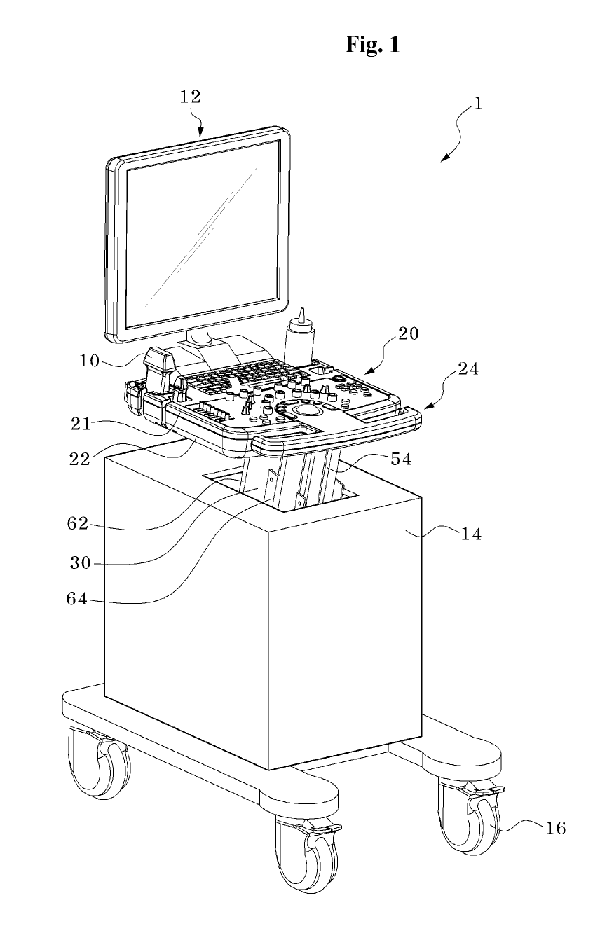

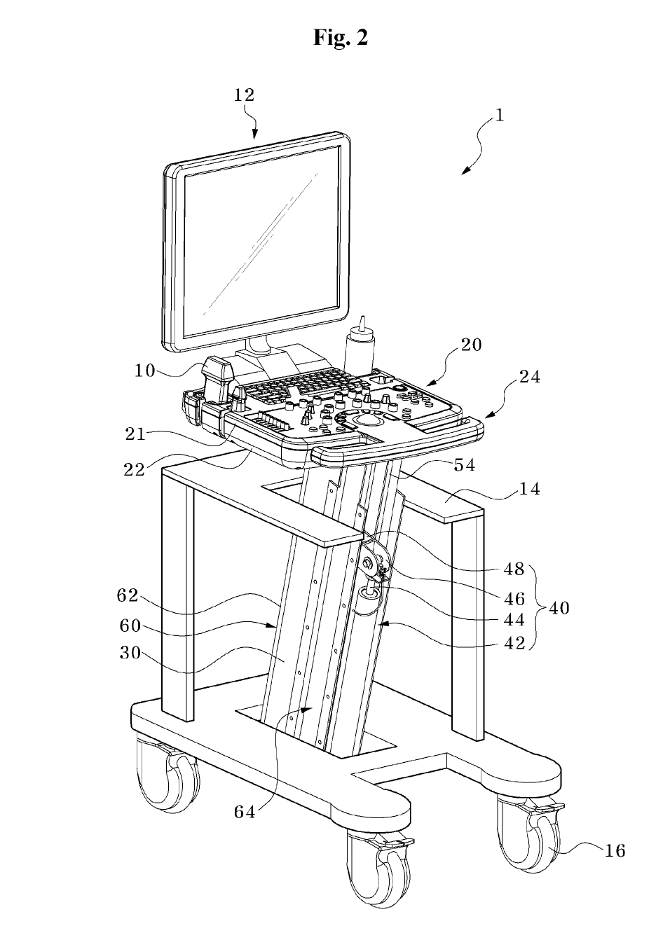

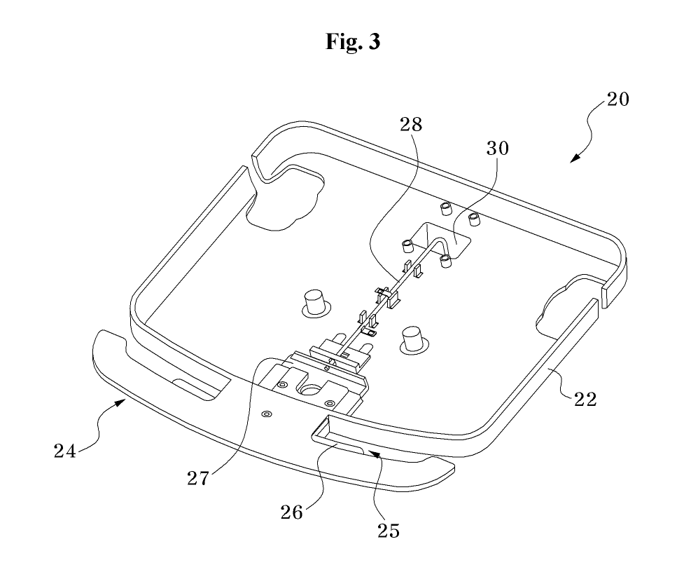

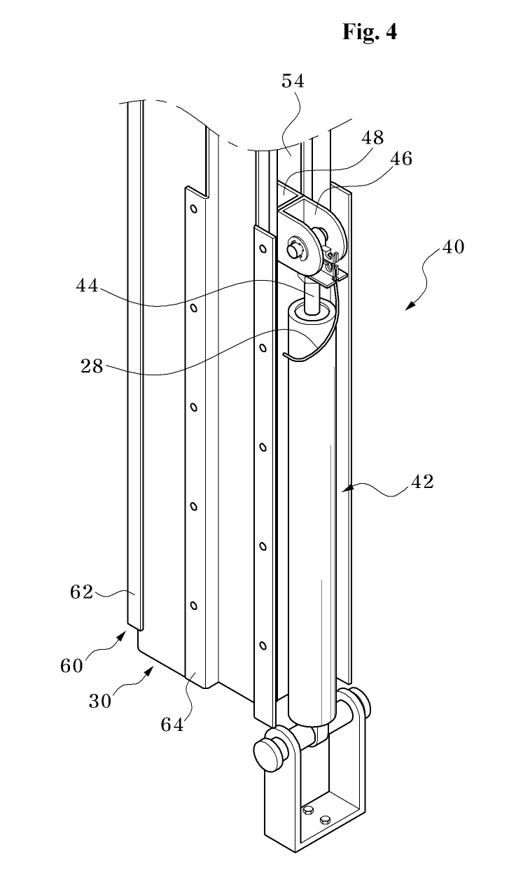

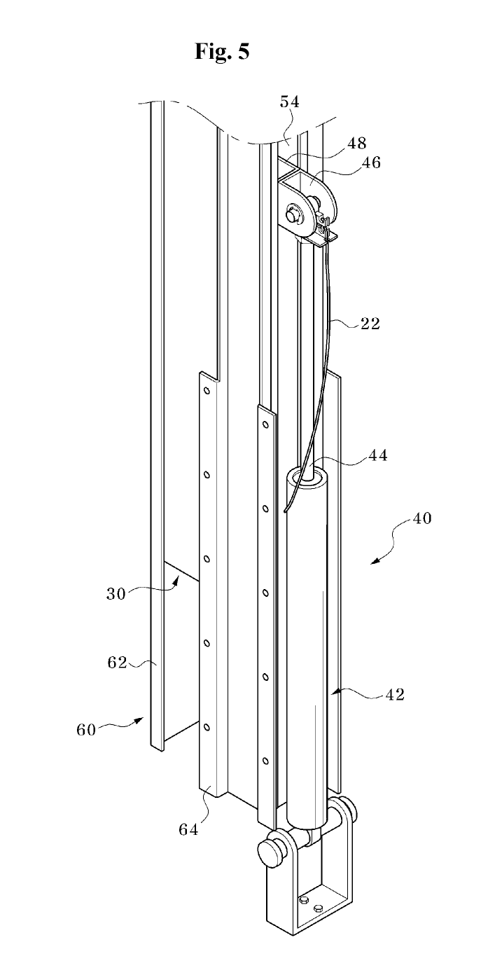

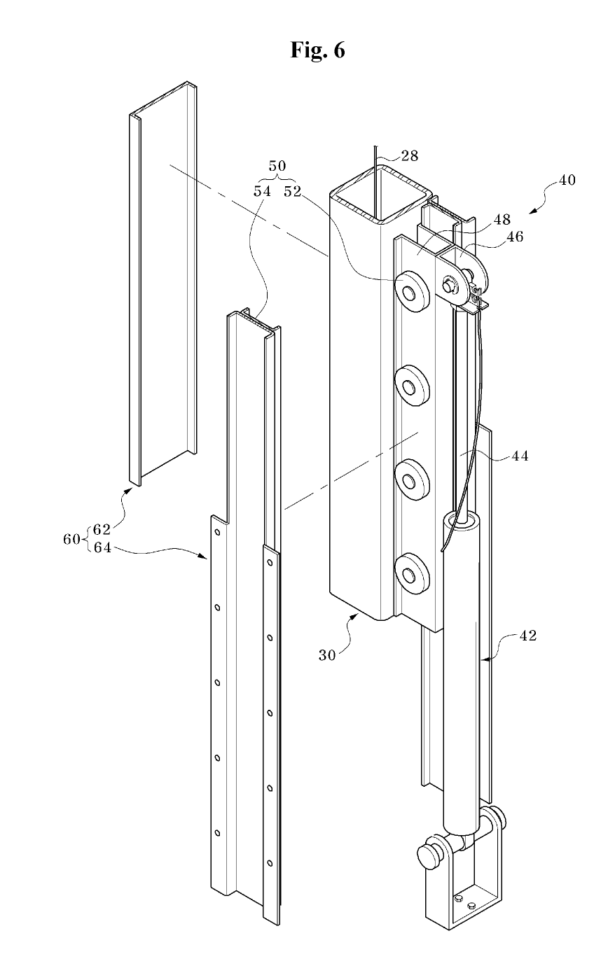

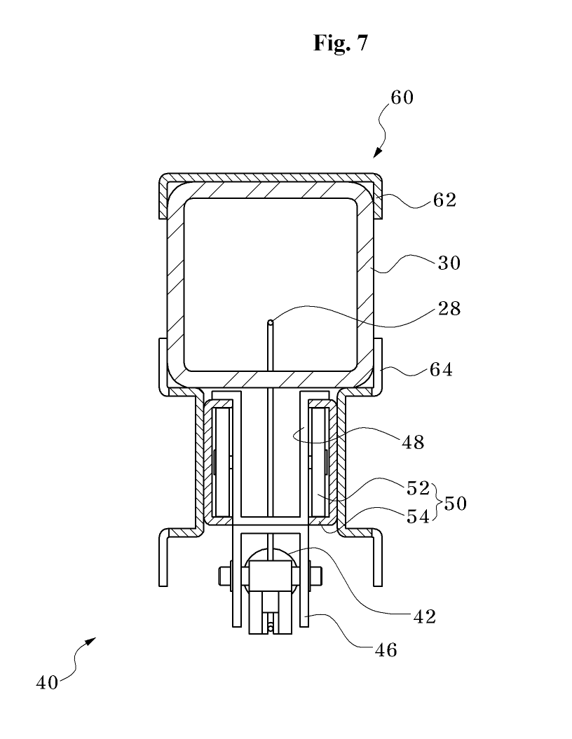

FIG. 1 is a schematic perspective view of an ultrasonic diagnostic apparatus including a variable elevation type control panel according to one embodiment, FIG. 2 is a schematic perspective view of a lift provided to the ultrasonic diagnostic apparatus according to the embodiment, FIG. 3 is a schematic perspective view of a lift operating part provided to the ultrasonic diagnostic apparatus according to the embodiment, FIG. 4 is a schematic perspective view of the lift before operation according to the embodiment; FIG. 5 is a schematic perspective view of the lift after operation according to the embodiment; FIG. 6 is an exploded perspective view of a rail member and a second guide part shown in FIG. 5; and FIG. 7 is a plan view of the lift provided to the ultrasonic diagnostic apparatus according to the embodiment.

Referring to FIGS. 1 to 3, the ultrasonic diagnostic apparatus 1 according to the embodiment includes a control panel 20 having a lift operating part 25, a support 30 supporting the control panel 20, and a lift 40 connected to the support 30 and operated by manipulation of the lift operating part 25 to lift or lower the support 30.

Referring to FIG. 2, an image display unit 12 is disposed above the control panel 20 to output an ultrasound image on a screen thereof.

The control panel 20 is configured to hold a probe 10 for ultrasound diagnosis therein and is connected at a lower side thereof to the support 30.

The support 30 supports the control panel 20 and may have any shape so long as the support can be lifted or lowered by operation of the lift 40. In one embodiment, the support 30 has a rectangular tube shape.

Since the lower side of the support 30 is secured to a main body 14 of the apparatus having wheels 16, the support 30 and the control panel 20 are also horizontally moved when the main body 14 is moved.

As shown in FIGS. 2 and 3, the control panel 20 includes upper and lower panels 21 and 22 coupled to each other and a gripper 24 protrudes forward from the control panel 20.

The gripper 24 is provided with the lift operating part 25 which controls operation of the lift 40.

The lift operation part 25 includes a gripping portion 26 protruding from the gripper 24, and an extended portion 27 connected to the gripping portion 26 and the cable 28 to move integrally with the gripping portion 26.

The gripping portion 26 is resiliently supported by and protruded from the gripper 24 to be moved into the lift operating part 25 when gripped by a user.

The cable 28 is moved by manipulation of the lift operating part 25 and is connected to a gas spring 42 of the lift 40 to lock or release the gas spring 42.

The lift 40 may adjust a position of the control panel 20 by raising or lowering the support 30 that supports the lower side of the control panel 20.

In one embodiment, the lift 40 includes the gas spring 42, which is connected to the lift operating part 25 via the cable 28 and adjusts a lifted or lowered position of the control panel 20, and a connecting member 48, which is connected at one side or both sides thereof to the support 30 and at the other side thereof to the gas spring 42.

In one embodiment, the gas spring 42 may provide a buffering function to absorb impact transferred to the control panel 20 and a locking function to secure the position of the control panel 20. Any kind of lifting device may be used so long as the lifting device can provide these functions.

The gas spring 42 relieves impact occurring when the support 30, control panel 20 and image display unit 12 are lifted or lowered. In particular, the gas spring 42 allows the control panel 20 to be lowered towards the ground at a constant speed, thereby providing stability.

Further, movement of a load member 44 is restricted by blocking a flow of a fluid inside the gas spring 42, so that a vertical movement of the control panel 20 associated with the movement of the load member 44 is also restricted.

The structure and operation of the gas spring 42 providing the buffering and locking functions is well known to those skilled in the art and a detailed description thereof will thus be omitted herein.

On the other hand, a lower side of the gas spring 42, first and second guide members 62 and 64, and a rail member 54 are secured to a lower side of the main body 14.

Referring to FIGS. 6 and 7, the connecting member 48 of the rectangular tube shape is secured along the support 30 and the load member 44 is connected to a connection bracket 46 disposed at one side or both sides of the connection member 48.

The connection member 48 is provided at one side or both sides thereof with a first guide part 50, which guides a vertical movement of the connection member 48.

In one embodiment, the first guide part 50 includes roller members 52 provided to one side or both sides of the connecting member 48, and a rail member 54 disposed along a movement path of the connecting member 48 while surrounding a side surface of each roller member 52.

The roller members 52 are disposed along the connection member 48, and the rail member 54 bent to surround the side surface of each roller member 52 extends vertically.

As for a second guide part 60 adjoining the support 30, any kind of guide device may be used so long as it can guide a vertical movement of the support 30.

Referring to FIG. 7, the second guide part 60 of one embodiment includes the first guide member 62 vertically disposed to surround a rear side of the support 30 and the second guide member 64 vertically disposed to surround a front edge of the support 30.

Next, operation of the ultrasonic diagnostic apparatus 1 with the variable elevation type control panel according to the embodiment will be described with reference to the accompanying drawings.

As shown in FIGS. 1 to 3, when changing the positions of the control panel 20 and the image display unit 12 in the vertical direction, an operator pulls the gripping portion 26, so that the extended portion 27 integrally formed with the gripping portion 26 is also moved.

As shown in FIGS. 4 and 5, the movement of the extended portion 27 causes the cable 28 connected to the load member 44 of the gas spring 42 to be pulled, so that the gas spring 42 is released from a locked state.

When the gas spring 42 is released from the locked state, an inner flow passage in the gas spring 42 is opened and an inner pressure imparted by a fluid flowing through the inner flow passage forces the load member 44 to move in an upward direction (when viewed in FIG. 5).

Then, in association with the load member 44, the connection member 48, the support 30 and the control panel 20 connected to the support 30 are also moved in the upward direction.

When the control panel 20 is lifted to a desired position, the operator releases the gripping portion 26 from a compressed state, so that the inner flow passage in the gas spring 42 is closed to maintain the load member 44 in a locked state, thereby restricting the movement of the load member 44.

That is, when lifting the control panel 20, the operator maintains the lift operating part 25 in a gripped state until the control panel 20 is automatically lifted to a desired position by force from the gas spring 42. Then, when the control panel 20 reaches the desired position, the operator releases the lift operating part 25 from the gripped state and fixes the position of the control panel 20.

When lowering the control panel 20, with the lift operating part 25 maintained in a gripped state, the operator compresses the control panel 20 with a greater force than an inner pressure of the gas spring 42, so that the control panel 20 is moved downward. Then, when the control panel 20 reaches a desired position, the operator releases the lift operating part 25 from the gripped state and fixes the position of the control panel 20.

The connection member 48 moved by operation of the gas spring 42 is guided upward or downward by the first guide part 50 and the support 30 is guided upward or downward by the second guide part 60, thereby providing more stable operation.

In the ultrasonic diagnostic apparatus 1 of the embodiment with the structure described above, the gas spring 42 operated by the lift operating part 25 adjusts the elevation of the control panel 20 when raising or lowering the control panel 20 and the image display unit 12 are required, thereby improving convenience in operation.

Further, instead of providing separate means to the control panel 20 to realize the impact buffering and locking functions, the gas spring 42 provides these functions, thereby reducing installation and maintenance costs.

Although some embodiments have been provided to illustrate the invention in conjunction with the drawings, it will be apparent to those skilled in the art that the embodiments are given by way of illustration only, and that various modifications and equivalent embodiments can be made without departing from the spirit and scope of the invention.

Further, although the gas spring is illustrated as being included in the lift in the above embodiments, it is obvious that this configuration is given by way of illustration only and any kind of position controller may be used for the lift of the present invention.

Therefore, the scope of the invention should be limited only by the accompanying claims and equivalents thereof

What is claimed is:

1. An ultrasonic diagnostic apparatus with variable elevation type control panel comprising:
a control panel having a lift operating part;
a support supporting the control panel; and
a lift connected to the support and operated by manipulation of the lift operating part to lift or lower the support.

2. The apparatus of claim 1, wherein the lift comprises a gas spring connected to the lift operating part via a cable and adjusting a lifted or lowered position of the control panel.

3. The apparatus of claim 2, wherein the lift further comprises a connecting member connected at one side thereof to the support and at the other side thereof to the gas spring.

4. The apparatus of claim 3, further comprising:
a first guide part provided to one side of the connecting member to guide a vertical movement of the connecting member.

5. The apparatus of claim 4, wherein the first guide part comprises a roller member provided to one side of the connecting member, and a rail member disposed along a movement path of the connecting member while surrounding a side surface of the roller member.

6. The apparatus of claim 1, further comprising:
a second guide part adjoining the support and guiding a vertical movement of the support.
